(19) United States
(12) Patent Application Publication
(10) Pub. No.: US 2012/0308621 A1
(43) Pub. Date: Dec. 6, 2012

(54) BASE FORMULATION FOR COSMETIC PRODUCTS

(76) Inventor: José Antonio NOVEJARQUE CONDE, Valencia (ES)

(21) Appl. No.: 13/491,918

(22) Filed: Jun. 8, 2012

Related U.S. Application Data
(63) Continuation of application No. PCT/ES2010/070803, filed on Dec. 3, 2010.

(30) Foreign Application Priority Data
Dec. 11, 2009 (ES) P200931147

(51) Int. Cl. A61K 8/92; A61Q 19/02; A61Q 15/00; A61Q 17/04; A61Q 1/02; A61Q 1/04; A61Q 1/10; A61Q 3/00; A61Q 5/06; A61Q 5/12; A61Q 11/00; A61Q 19/08; A61K 31/015; A61K 8/97; A61K 8/14; A61Q 13/00; A61Q 9/04; A61Q 5/10; A61Q 5/08; A61Q 5/02; A61Q 9/00; A61Q 5/00

(52) U.S. Cl. 424/401; 514/772.4; 424/70.11; 424/62; 424/65; 424/59; 424/63; 424/64; 424/70.7; 424/61; 424/49; 514/763; 424/725; 512/1; 8/161; 8/405; 510/119; 424/73

(57) ABSTRACT

The present invention relates to a base formulation for cosmetic products, characterized in that it comprises, in percentage by weight of the formulation and according to the INCI nomenclature:
a) from 0.2 to 10% of at least one hydroxylated ester;
b) from 0.5 to 70% of a mixture of at least one mineral oil and at least one copolymer derived from alkenes;
c) from 0.5 to 90% of at least one hydrogenated polymer;
d) from 0.5 to 35% of a mixture of water and at least one glyceryl glucoside.

Likewise, an object of this invention is a cosmetic product characterized in that it comprises the aforementioned base formulation, as well as the preparation procedure of the same.

### TECHNICAL FIELD

The present invention is framed within the field of cosmetics. Especially, it relates to a new base formulation for cosmetic products, characterized in that they present different degrees of texture and in that they are completely transparent.

### STATE OF THE ART PRIOR TO THE INVENTION

In the last few years, a great variety of cosmetic products have been developed based on oils and gelling agents. In ES 2321495, for example, a gel-type product based on this type of compounds with improved structural and stability characteristics is presented.

One of the characteristics of the cosmetic products obtained from the formulation according to the present invention is their transparent or translucent appearance.

In literature, we can find patents such as, for example, ES 2125482, whose object of protection is aimed at a cosmetic product comprising a clear, single-phase composition. On the other hand, ES 2086932 describes a cosmetic composition for makeup, characterized by containing a transparent, titanium oxide and silicon oxide pigment.

The object of this invention is to present a new base formulation for cosmetic products, characterized in that they present different degrees of texture and in that they are completely transparent. Generally, the viscosity of the final product will be relatively high and, in addition, it will exhibit a correct binding and homogenization of all the compounds present thereof, having, in turn, a practically insignificant water percentage.

### DESCRIPTION OF THE INVENTION

The present invention relates to, therefore, a new base formulation for cosmetic products, characterized by comprising, in percentage by weight of the formulation and according to the INCI nomenclature:
a) from 0.2 to 10%, preferably from 0.5 to 6%, of at least one hydroxylated ester, preferably, PEG-20 glyceryl triisostearate, such as, for example , NIKKOL TGI-20, sold by Nikko Chemicals Co. Ltd.;
b) from 0.5 to 70%, preferably from 1 to 60%, of a mixture of at least one mineral oil and at least one copolymer derived from alkenes, preferably selected from a group consisting of an ethylene, propylene and styrene copolymer and a butylene, ethylene and styrene copolymer, as well as any combination thereof. This mixture may consist, for example, of VERSAGEL M-750, sold by Calumet Product Partners L.P.;
c) from 0.5 to 90%, preferably from 1 to 80%, of at least one hydrogenated polymer, preferably, at least one polyalphaolefin, and more preferably, hydrogenated dodecene, such as, for example NEXBASE 2002, sold by Neste Oil;
d) from 0.5 to 35%, preferably from 1 to 25%, of a mixture of water and at least one glyceryl glucoside, such as, for example, GLYCOIN EXTREMIUM, sold by Jan Dekker Int.

The combination of the aforementioned compounds allows obtaining a formulation with optimal moisturizing and emollient characteristics, presenting a viscosity of 70.1 to 100,000 cps, where the minimum value corresponds to the viscosity of a conventional olive oil.

Preferably, the previous formulation may also comprise, in the percentage by weight of the formulation and according to the INCI nomenclature, at least one additional ingredient selected from a group consisting of:
e) a polysaccharide in a percentage of 0.05 to 8%, preferably from 0.1 to 3%, preferably selected between Chitosan and Beta-glucan, and any combination thereof, such as, for example, KIOSMETINE CG 125, sold by KitoZyme SA;
f) a natural extract in a percentage from 0.5 to 10%, preferably from 1 to 5%, preferably selected between an extract of Jojoba seed oil (Simmonidisia chinensis) and an extract of the fruit of Opuntia Ficus Indica, as well as any combination thereof, such as, for example, REPARAMI, sold by Alban Muller Group Int.; and
g) a light medicinal oil in a percentage from 0.5 to 95%, preferably from 1 to 90%, and said medicinal oil is preferably constituted by a mixture of paraffinic and naphthenic hydrocarbons and is free from aromatic hydrocarbons, such as, for example, the one sold by QUIMIDROGA S.A.;
   as well as any combination thereof.

This way, the compounds comprising the formulation may consist of synthetic, natural or ecologic compounds.

Likewise, the formulation according to the invention may comprise another series of compounds selected from the ones normally used in the manufacturing of composite products. These compounds may be preferably selected, in a non-limiting manner, from a group consisting of odoriferous (perfumes, fragrances or aromas), stabilizing, pH-regulating and antioxidant substances, biocides and conservatives, medicinal, soothing, anti-cellulite, anti-wrinkle and anti-dandruff substances, hair straighteners, detangling, reaffirming, anti-corrosive, chelating, anti-foam, solvent, thickening, gelling, emollient, emulsifying, anti-binding, surfactant (anionic, cationic, amphoteric or non-ionic), emulsifying, exfoliating, moisturizing, humectant, makeup-removing, pigment-removing, pearlizing, solvent, deodorizing, antiperspirant, tanning, colouring, opacifying, whitening and bleaching substances, as well as any combination thereof.

The aforementioned compounds may consist, for example, of vitamins, emollient esters, lanolins, UV filters or protectors (antioxidant, organic or inorganic), vegetable oils, medicinal, pharmaceutical or cosmetic mineral oils (paraffinic, naphthenic or aromatic), essential oils, biological products, galenic products, plant extracts (flower or fruit extracts, dry extracts, glycolic, hydroglycolic, oily, colourless and water-soluble extracts), amino acids, alcohols, glycerines and vegetable glycerines, olive oil derivatives, vegetable petrolatums, silicones, fatty acids, oligo-elements, proteins, peptides, liposomes, active organic oils, natural active ingredients from vegetable, sea or synthetic origin, Aloe Vera and lipids, as well as any combination thereof.

Likewise, the object of this invention is a cosmetic product that comprises the aforementioned formulation. This product will be characterized by being hypoallergenic and non-irritating, as well as by presenting a transparent or translucent appearance, depending on the raw materials used in its manufacturing. Regarding the viscosity values of this cosmetic product, they are comprised between 70.1 y 100.000 cps.

According to another embodiment of the invention, the final cosmetic product may comprise from 0 to 7% by weight of water, without affecting its final properties. Said percentage may be pure deionized, osmotic and/or mineral water added, or it may come implicitly as a subcomponent of any of the compounds making up the formulation of the invention.

The cosmetic products according to the invention may be used in very diverse applications, such as, for example, the manufacturing of skin creams, emulsions, lotions, gels and oils; beauty masks (excluding chemical surface abrasion products for the skin); makeup (liquid, paste and powder); makeup powders, styling powder for after bathing and for body hygiene; beauty soap and deodorant soap; perfumes, eau de toilette and colognes; bath and shower products (salts, foams, oils and gels); depilatory products; deodorants and antiperspirants; hair products such as hair dyes and bleaches, products for moulding, straightening and setting hair, products that help maintaining the hairstyle, cleaning products (lotions, powders or shampoos), conditioning products (lotions or brilliantine hair sprays) and other hairstyling products; shaving products (soaps, foams, gels or lotions); face and eye makeup and makeup-removing products; lip products; mouth and teeth care products; nail makeup and care products; external intimate care products; sun care products; sunless tanning products; skin whitening or anti-wrinkle products.

Likewise, particularly, it may be used in the manufacturing of decorative-type cosmetic products such as, for example, lip makeup, eye shadow, blush, eyeliner, fluid makeup, powder makeup, mascara or nail polish.

Likewise, an additional object of this invention is the procedure to prepare the aforementioned formulation for cosmetic products. This procedure comprises, at least, the following steps:
a) mixing, while stirring continuously, the components of the formulation, excepting the mixture of at least one mineral oil and at least one copolymer derived from alkenes, until obtaining a complete and homogeneous mixture;
b) heating the aforementioned mixture to a temperature between 50 and 90° C.;
c) adding the mixture of at least one mineral oil and at least one copolymer derived from alkenes to the aforementioned mixture, while stirring the mixture continuously during a period of time between 20 and 60 min;
d) cooling the aforementioned mixture to room temperature.

In a preferred embodiment of the invention, the aforementioned procedure will also comprise an additional packaging step for the formulation, in the desired dosed amounts.

Likewise, in a preferred embodiment in which the formulation of the invention comprises, additionally, at least one light medicinal oil, the same will be added to the manufacturing reactor after being weighted in an additional step prior to the addition of the remaining components.

### PREFERRED EMBODIMENT OF THE INVENTION

We will now present, as an example and in a non-limiting manner, the description of a preferred embodiment of the present invention.

This way, a formulation was prepared according to the following percentages by weight:

NIKKOL TGI-20: 3%

KIOSMETINE CG. 125: 3%

VERSAGEL M-750: 50%

ECOLOGICAL ARGAN OIL: 30%

GLYCOINT™ EXTREMIUM: 4%

NEXBASE 2002: 10%

In the first place, the ecological argan oil was weighted and poured into the manufacturing reactor. Afterwards, with the reactor stirring at a speed of 100 to 500 rpm and at a temperature of 40 to 85° C., the remaining components were incorporated (NIKKOL TGI-20, KIOSMETINE CG. 125, GLYCOIN EXTREMIUM and NEXBASE 2002). Finally, VERSAGEL M-700 was slowly incorporated at a temperature of approximately 85° C. in a homogeneous manner and by stirring continuously.

The final product obtained, aside from presenting a correct binding and homogenization of all the components of the formulation, allowed obtaining very satisfactory results, both for regenerating and nourishing the skin.

1. Base formulation for cosmetic products, characterized in that it comprises, in percentage by weight of the formulation and according to the INCI nomenclature:
a) from 0.2 to 10% of at least one hydroxylated ester;
b) from 0.5 to 70% of a mixture of at least one mineral oil and at least one copolymer derived from alkenes;
c) from 0.5 to 90% of at least one hydrogenated polymer;
d) from 0.5 to 35% of a mixture of water and at least one glyceryl glucoside.

2. Formulation according to claim 1, wherein the hydroxylated ester consists of PEG-20 glyceryl triisostearate.

3. Formulation according to claim 1, wherein the mixture of at least one mineral oil and at least one copolymer derived from alkenes consists of a mixture of mineral oil and at least one copolymer derived from alkenes selected from a group consisting of ethylene, propylene and styrene copolymer and a butylene, ethylene and styrene copolymer, as well as any combination thereof.

4. Formulation, according to claim 1, wherein the hydrogenated polymer consists of a polyalphaolefin.

5. Formulation, according to claim 1, characterized in that it comprises, in percentage by weight of the formulation and according to the INCI nomenclature, at least one additional ingredient, selected from a group consisting of:
a) a polysaccharide in a percentage of 0.05 to 8%, preferably selected between Chitosan and Beta-glucan, as well as any combination thereof;
b) a natural extract in a percentage from 0.5 to 10%, preferably selected between an extract of Jojoba seed oil (Simmonidisia chinensis) and an extract of the fruit of Opuntia ficus indica, as well as any combination thereof; and
c) a light medicinal oil in a percentage from 0.5 to 95%,
as well as any combination of the previous ingredients.

6. Formulation, according to claim 1, characterized in that it also comprises at least one additional compound selected from a group consisting of odoriferous, stabilizing, pH-regulating and antioxidant substances, biocides and conservatives, medicinal, soothing, anti-cellulite, anti-wrinkle and anti-dandruff substances, hair straighteners, detangling, reaffirming, anti-corrosive, anti-foam, solvent, thickening, chelating, gelling, emollient, emulsifying, anti-binding, surfactant, emulsifying, exfoliating, moisturizing, humectant, makeup-removing, pigment-removing, solvent, deodorizing, antiperspirant, colouring, pearlizing, opacifying, tanning, whitening and bleaching substances, as well as any combination thereof.

7. Formulation according to claim 6, in which said compound is selected from a group consisting of vitamins, emollient esters, lanolins, UV protectors, vegetable oils, mineral oils for medicinal, pharmaceutical or cosmetic use, essential oils, biological products, galenic products, plant extracts, amino acids, alcohols, glycerines, olive oil derivatives, vegetable petrolatums, silicones, fatty acids, oligo elements, proteins, peptides, liposomes, active organic oils, natural active ingredients from vegetable, sea or synthetic origin, Aloe Vera and lipids, as well as any combination thereof.

8. Cosmetic product characterized in that it comprises a formulation according to claim 1.

9. Use of a cosmetic product, according to claim 8, for the manufacturing of at least one product selected from skin creams, emulsions, lotions, gels and oils; beauty masks excluding chemical surface abrasion products for the skin; makeup; makeup powders, styling powder for after bathing and for body hygiene; beauty soap and deodorant soap; perfumes, eau de toilette and colognes; bath and shower products; depilatory products; deodorants and antiperspirants; hair products such as hair dyes and bleaches, products for moulding, straightening and setting hair, products that help maintaining the hairstyle and hair washing or conditioning products; shaving products; face and eye makeup and makeup-removing products; lip products; mouth and teeth care products; nail makeup and care products; external intimate care products; sun care products; sunless tanning products; skin whitening or anti-wrinkle products.

10. Use of a cosmetic product according to claim 8, for the manufacturing of a decorative-type cosmetic product selected from a group consisting of lip makeup, eye shadow, blush, eyeliner, fluid makeup, powder makeup, mascara and nail polish, as well as any combination thereof.

11. Preparation procedure for a formulation according to claim 1, characterized by comprising, at least, the following steps:
a) mixing, while stirring continuously, the components of the formulation, excepting the mixture of at least one mineral oil and at least one copolymer derived from alkenes until obtaining a complete and homogeneous mixture;
b) heating of the aforementioned mixture to a temperature between 50 and 90 ° C.;
c) adding the mixture of at least one mineral oil and at least on copolymer derived from alkenes to the aforementioned mixture, while stirring the mixture continuously during a period of time between 20 and 60 min;
d) cooling the aforementioned mixture to room temperature.

12. Procedure, according to claim 11, characterized by comprising an additional initial step to add at least one light medicinal oil.
